(19) United States
(12) Patent Application Publication
(10) Pub. No.: US 2006/0166212 A1
(43) Pub. Date: Jul. 27, 2006

(54) Breast specific protein expressed in cancer and methods of use thereof

(76) Inventors: Bernd Weigle, Hanau (DE); Michael Rieger, Dresden (DE); Reinhard Ebner, Gaitherburg, MD (US)

Correspondence Address: CARELLA, BYRNE, BAIN, GILFILLAN, CECCHI,;STEWART & OLSTEIN, 5 BECKER FARM ROAD, ROSELAND, NJ, 07068, US

(21) Appl. No.: 10/540,310

(22) PCT Filed: Dec. 19, 2003
(86) PCT No.: PCT/US03/40710

§ 371 Date: Dec. 12, 2005

Related U.S. Application Data
(60) Provisional application No. 60/434,960, filed on Dec. 20, 2002.

(51) Int. Cl. C12Q 1/68

(52) U.S. Cl. 435/6

(57) ABSTRACT

Cancer-linked gene sequences, and derived amino acid sequences, are disclosed along with processes for assaying potential antitumor agents based on their modulation of the expression of these cancer-linked genes. Also disclosed are antibodies that react with the disclosed polypeptides and methods of using the antibodies to treat cancerous conditions, such as by using the antibody to target cancerous cells in vivo for purposes of delivering therapeutic agents thereto. Also described are methods of diagnosing using the gene sequences.

This application claims priority of U.S. Provisional Application Ser. No. 60/434,960, filed 20 Dec. 2002, the disclosure of which is hereby incorporated by reference in its entirety.

### FIELD OF THE INVENTION

The present invention relates to CYP4Z1, a new breast specific protein belonging to the cytochrome P450 family and its potential use for the screening, diagnosis, and treatment of breast cancer, as well as the use of such protein for screening potential anti-cancer agents, including small organic compounds and other molecules, and development of therapeutic agents.

### BACKGROUND OF THE INVENTION

Cancer-linked genes are valuable in that they indicate genetic differences between cancer cells and normal cells, such as where a gene is expressed in a cancer cell but not in a non-cancer cell, or where said gene is over-expressed or expressed at a higher level in a cancer as opposed to normal or non-cancer cell. In addition, the expression of such a gene in a normal cell but not in a cancer cell, especially of the same type of tissue, can indicate important functions in the cancerous process. For example, screening assays for novel drugs are based on the response of model cell based systems in vitro to treatment with specific compounds; Such genes are also useful in the diagnosis of cancer and the identification of a cell as cancerous. Gene activity is readily measured by measuring the rate of production of gene products, such as RNAs and polypeptides encoded by such genes. Where genes encode enzyme proteins, changes in the enzyme activity, including increased expression or change in activity due to mutation, are an indication of neoplastic activity. In addition, where said polypeptides represent cell surface antigens, available for detection or modulation through specific agents, such as antibodies, such as antibodies complexed with a cytotoxic agent, such as an apoptotic agent, the presence or absence of such antigen, or increased expression thereof, is useful for diagnosis and treatment of cancerous conditions. In accordance with the present invention, a cancer-linked gene has been identified and its putative amino acid sequence worked out. Such gene is over-expressed in breast cancer cells and is useful in the diagnosing of cancer, the screening of anticancer agents and the treatment of cancer using such agents, especially in that this gene encodes an enzyme that provides a ready target for anti-tumor agents, including both antibodies that may bind to the protein as well as agents, such as small organic compounds, that modulate, the activity of the enzyme, such as where the activity is correlated with cancerous characteristics and the agent serves to inhibit enzyme activity and thereby ameliorate, or prevent, the cancerous condition.

Such advantages are afforded by the present invention, which discloses CYP4Z1, a new breast specific protein belonging to the cytochrome P450 family and its potential use for the screening, diagnosis, and treatment of breast cancer.

### BRIEF SUMMARY OF THE INVENTION

In accordance with the present invention, there is provided herein a cancer specific gene, linked especially to breast cancer, or otherwise involved in the cancer initiating and facilitating process and the derived amino acid sequence thereof, including a polypeptide encoded by said gene (and the cDNA encoding said polypeptide), which polypeptide has cytochrome P450 oxidase activity.

In one aspect, the present invention relates to a process for identifying an agent that modulates the activity of a cancer-related gene comprising contacting a compound with a cell containing such a gene and under conditions promoting the expression of said gene and then determining a difference in expression of said gene relative to when said compound is not present thereby identifying an agent that modulates the activity of a cancer-related gene. In various embodiments of such a process, the cell is a cancer cell and the difference in expression is a decrease in expression.

In another aspect, the present invention relates to a process for identifying an anti-neoplastic agent comprising contacting a cell exhibiting neoplastic activity with a compound first identified as a cancer related gene modulator using an assay process disclosed herein and detecting a decrease in said neoplastic activity after said contacting compared to when said contacting does not occur.

The present invention also relates to a process for identifying an anti-neoplastic agent comprising administering to an animal exhibiting a cancer condition an effective amount of an agent first identified according to a process of one of one of the assays disclosed according to the invention and detecting a decrease in said cancerous condition.

The present invention further relates to a process for determining the cancerous status of a cell, comprising determining an increase in the level of expression in said cell of a cancer-specific gene wherein an elevated expression relative to a known non-cancerous cell indicates a cancerous state or potentially cancerous state. Such elevated expression may be due to an increased copy number.

The present invention additionally relates to an isolated polypeptide, encoded by one of the polynucleotide transcripts disclosed herein, comprising an amino acid sequence homologous to the amino acid sequence of SEQ ID NO: 4.

The present invention also relates to an antibody that reacts with a polypeptide as disclosed herein, preferably a polypeptide comprising the amino acid sequence of SEQ ID. NO: 4. Such an antibody may be polyclonal, monoclonal, recombinant or synthetic in origin.

In one such embodiment, said antibody is associated, either covalently or non-covalently, with a cytotoxic agent, for example, an apoptotic agent. Thus, the present invention relates to an immunoconjugate comprising an antibody of the invention and a cytotoxic agent.

The present invention also relates to a process for treating cancer comprising contacting a cancerous cell with an agent having activity against an expression product encoded by a cancer-specific gene sequence as disclosed herein.

The present invention further encompasses an immunogenic composition comprising a polypeptide disclosed herein, as well as compositions formed using antibodies specific for these polypeptides.

The present invention is also directed to uses of such compositions. Such uses include a method for treating cancer in an animal afflicted therewith comprising administering to said animal an amount of an immunogenic composition of one or more of the polypeptides disclosed herein where such amount is an amount sufficient to elicit the production of cytotoxic T lymphocytes specific for a polypeptide of the invention.

### Definitions

“CYP4Z1” is a new breast-specific transcript that is expressed in female breast tissue and is upregulated in breast cancer. It encodes a protein belonging to the family of cytochrome P450 oxidases and is therefore predicted to have enzymatic activity. Because of its highly specific expression in female breast tissue, it is an excellent target candidate for cell-based anti-cancer immunotherapy.

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. When used in relation to a polynucleotides, such terms refer to the products produced by treatment of said polynucleotides with any of the common endonucleases.

As used herein, the term “isolated” means that the material is removed from its original environment (e.g., the natural environment if it is naturally occurring). It could also be produced recombinantly and subsequently purified. For example, a naturally-occurring polynucleotide or polypeptide present in a living animal is not isolated, but the same polynucleotide or polypeptide, separated from some or all of the coexisting materials in the natural system, is isolated. Such polynucleotides, for example, those prepared recombinantly, could be part of a vector and/or such polynucleotides or polypeptides could be part of a composition, and still be isolated in that such vector or composition is not part of its natural environment. In one embodiment of the present invention, such isolated, or purified, polypeptide is useful in generating antibodies for practicing the invention, or where said antibody is attached to a cytotoxic or cytolytic agent, such as an apoptotic agent.

The term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:
Percent Identity=100[1−(C/R)]
wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

As known in the art “similarity” between two polypeptides is determined by comparing the amino acid sequence and its conserved amino acid substitutes of one polypeptide to the sequence of a second polypeptide.

In accordance with the present invention, the term “DNA segment” or “DNA sequence” refers to a DNA polymer, in the form of a separate fragment or as a component of a larger DNA construct, which has been derived from DNA isolated at least once in substantially pure form, i.e., free of contaminating endogenous materials and in a quantity or concentration enabling identification, manipulation, and recovery of the segment and its component nucleotide sequences by standard biochemical methods, for example, using a cloning vector. Such segments are provided in the form of an open reading frame uninterrupted by internal nontranslated sequences, or introns, which are typically present in eukaryotic genes. Sequences of non-translated DNA may be present downstream from the open reading frame, where the same do not interfere with manipulation or expression of the coding regions.

The term “coding region” refers to that portion of a gene which either naturally or normally codes for the expression product of that gene in its natural genomic environment, i.e., the region coding in vivo for the native expression product of the gene. The coding region can be from a normal, mutated or altered gene, or can even be from a DNA sequence, or gene, wholly synthesized in the laboratory using methods well known to those of skill in the art of DNA synthesis.

In accordance with the present invention, the term “nucleotide sequence” refers to a heteropolymer of deoxyribonucleotides. Generally, DNA segments encoding the proteins provided by this invention are assembled from cDNA fragments and short oligonucleotide linkers, or from a series of oligonucleotides, to provide a synthetic gene which is capable of being expressed in a recombinant transcriptional unit comprising regulatory elements derived from a microbial, eukaryotic or viral operon.

The term “expression product” means that polypeptide or protein that is the natural translation product of the gene and any nucleic acid sequence coding equivalents resulting from genetic code degeneracy and thus coding for the same amino acid(s).

The term “active fragment,” when referring to a coding sequence, means a portion comprising less than the complete coding region whose expression product retains essentially the same biological function or activity as the expression product of the complete coding region.

The term “primer” means a short nucleic acid sequence that is paired with one strand of DNA and provides a free 3′-OH end at which a DNA polymerase starts synthesis of a deoxyribonucleotide chain.

The term “promoter” means a region of DNA involved in binding of RNA polymerase to initiate transcription. The term “enhancer” refers to a region of DNA that, when present and active, has the effect of increasing expression of a different DNA sequence that is being expressed, thereby increasing the amount of expression product formed from said different DNA sequence.

The term “open reading frame (ORF)” means a series of triplets coding for amino acids without any termination codons and is a sequence (potentially) translatable into protein.

As used herein, reference to a “DNA sequence” includes both single stranded and double stranded DNA. Thus, the specific sequence, unless the context indicates otherwise, refers to the single strand DNA of such sequence, the duplex of such sequence with its complement (double stranded DNA) and the complement of such sequence.

As used herein, “corresponding genes” refers to genes that encode an RNA that is at least 90% identical, preferably at least 95% identical, most preferably at least 98% identical, and especially identical, to an RNA encoded by one of the nucleotide sequences disclosed herein (i.e., SEQ ID NO: 1, 2, 3 and 4). Such genes will also encode the same polypeptide sequence as any of the sequences disclosed herein, preferably SEQ ID NO: 1, 2, 3 and 4, but may include differences in such amino acid sequences where such differences are limited to conservative amino acid substitutions, such as where the same overall three dimensional structure, and thus the same antigenic character, is maintained. Thus, amino acid sequences may be within the scope of the present invention where they react with the same antibodies that react with polypeptides comprising the sequences of SEQ ID NO: 5 and 6. A “corresponding gene” includes splice variants thereof.

The genes identified by the present disclosure are considered “cancer-related” genes, as this term is used herein, and include genes expressed at higher levels (due, for example, to elevated rates of expression, elevated extent of expression or increased copy number) in cancer cells relative to expression of these genes in normal (i.e., non-cancerous) cells where said cancerous state or status of test cells or tissues has been determined by methods known in the art, such as by reverse transcriptase polymerase chain reaction (RT-PCR) as described in the Examples herein. In specific embodiments, this relates to the genes whose sequences correspond to the sequences of SEQ ID NO: 1, 2, 3 and 4.

As used herein, the term “conservative amino acid substitution” is defined as an exchange within one of the following five groups:

I. Aliphatic, nonpolar or slightly polar residues:
Ala, Ser, Thr, Pro, Gly;

II. Negatively charged residues and their amides:
Asp, Asn, Glu, Gin;

III. Positively charged residues:
His, Arg, Lys;

IV. Large, aliphatic, nonpolar residues:
Met Leu, Ile, Val, Cys

V. Aromatic residues:
Phe, Tyr, Trp

### DETAILED SUMMARY OF THE INVENTION

The present invention relates to processes for utilizing a nucleotide sequence for a cancer-linked gene, polypeptides encoded by such sequences and antibodies reactive with such polypeptides in methods of treating and diagnosing cancer, preferably breast cancer, and in carrying out screening assays for agents effective in reducing the activity of cancer-linked genes and thereby treating a cancerous condition.

In accordance with the present invention, CYP4Z1 is a new breast-specific transcript that is expressed in female breast tissue and is upregulated in breast cancer. It encodes a protein belonging to the family of cytochrome P450 oxidases and is therefore predicted to have enzymatic activity. Because of its highly specific expression in female breast tissue, it is a useful target for cell-based anti-cancer immunotherapy. This polypeptide has the sequence shown in SEQ ID NO: 4, with the corresponding cDNA shown as SEQ ID NO: 3.

The present application discloses polynucleotides (including two known EST sequences with Accession Numbers A1668602 (SEQ ID NO: 1) and AV700083—SEQ ID NO: 2) and a polypeptide (CYP4Z1—SEQ ID NO: 4) with its encoding cDNA transcript (SEQ ID NO: 3) useful in the methods of the invention.

CYP4Z1 cDNA (SEQ ID NO: 3 from breast-specific mRNA) is useful for screening for disseminated tumor cells by PCR, and immunotherapy of breast cancer using transfected antigen-presenting cells. CYP4Z1 protein (SEQ ID NO: 4) is useful as a breast specific antigen, for screening for disseminated tumor cells by antibody, and for immunotherapy of breast cancer using protein or related peptides for induction of CD4+ cells and CD8+ cells. The expression and activity of this polypeptide, as an indicator of the cancerous state and for screening for antitumor agents, can be determined by a measure of P450 oxidase activity.

In a GX2000 screen for genes specifically expressed in female breast tissue the EST A1668602 (95 k chip) was identified. Supporting this finding, the EST AV700083, representing almost the same sequence as A1668602, is specifically expressed in female breast tissue when using the 133 k chip.

Electronic northern provided evidence for expression in about 80% of both malignant and nonmalignant breast tissue specimens, the median level of expression increasing from 165 FU (relative fluorescence units) in normal tissue to 213 FU in cancer tissue. Noteworthy, a fraction of the breast cancer tissues demonstrated expression several factors above this level. Background expression in all available normal tissues was almost undetectable.

Experimental evidence for breast-specific expression was provided by quantitative reverse transcription PCR using a multiple housekeeping gene standardized cDNA panel representing 16 normal tissues (BD Clontech) and CYP4Z1 cDNA specific primers. Expression was highest in breast (21.6 arbitrary units), followed by prostate (1.7 a.u.), testis (1.6 a.u.), lung (1.4 au), and liver (1 a.u.).

Further experimental evidence was provided by hybridizing a radioactive probe representing CYP4Z1 cDNA to a cancer profiling array (BD Clontech), representing cDNAs of more than 200 samples from paired normal/tumor tissue samples originating from 12 different organs. Remarkably, almost all of the breast cancer tissue samples showed expression, including three metastases originating from breast cancer.

Using a combination of data-mining (custom EST clustering) and 5′-RACE and 3′-RACE, the A1668620/AV700083 sequence was extended to full length cDNA, coding for a protein with homology to cytochrome P450 oxidases (CYP). From sequence of mouse and rat CYP, different researchers in the field of CYP had assumed that a yet unidentified human homolog had to exist. The CYP we identified is homologous with mouse and rat CYP, and we therefore conclude that it is indeed the predicted CYP4Z1.

The sequences disclosed herein incorporate the polynucleotide transcript of SEQ ID NO: 3 and the derived amino acid sequence (SEQ ID NO: 4) from said transcripts are available as targets for chemotherapeutic agents, especially anti-cancer agents, including antibodies specific for said polypeptides. The transcript for this gene is up in breast cancer and encodes a cytochrome P450 oxidase.

The cancer-related polynucleotide sequences disclosed herein correspond to gene sequences whose expression is indicative of the cancerous status of a given cell. Such sequences are substantially identical to SEQ ID NO: 3, which represents a transcript identified from the GenBank EST database and which exhibits cancer-specific expression. The polynucleotides of the invention are those that correspond to the sequence of SEQ ID NO: 3.

The nucleotides and polypeptides, as gene products, used in the processes of the present invention may comprise a recombinant polynucleotide or polypeptide, a natural polynucleotide or polypeptide, or a synthetic polynucleotide or polypeptide, or a recombinant polynucleotide or polypeptide.

Fragments of such polynucleotides and polypeptides as are disclosed herein may also be useful in practicing the processes of the present invention. For example, a fragment, derivative or analog of the polypeptide (SEQ ID NO: 4) may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as a leader or secretory sequence or a sequence which is employed for purification of the mature polypeptide (such as a histidine hexapeptide) or a proprotein sequence. Such fragments, derivatives and analogs are deemed to be within the scope of those skilled in the art from the teachings herein.

In another aspect, the present invention relates to an isolated polypeptide, including a purified polypeptide, comprising an amino acid sequence at least 90% identical to the amino acid sequence of SEQ ID NO: 4. In preferred embodiments, said isolated polypeptide comprises an amino acid sequence having sequence identity of at least 95%, preferably at least about 98%, and especially is identical to, the sequence of SEQ ID NO: 4. The present invention also includes isolated active fragments of such polypeptides where said fragments retain the biological activity of the polypeptide or where such active fragments are useful as specific targets for cancer treatment, prevention or diagnosis. Thus, the present invention relates to any polypeptides, or fragments thereof, with sufficient sequence homology to the sequences disclosed herein as to be useful in the production of antibodies that react with (i.e., are selective or specific for) the polypeptide of SEQ ID NO: 4 so as to be useful in targeting cells that exhibit such polypeptides, or fragments, on their surfaces, thereby providing targets for such antibodies and therapeutic agents associated with such antibodies.

The polynucleotides and polypeptides useful in practicing the processes of the present invention may likewise be obtained in an isolated or purified form. In addition, the polypeptide disclosed herein as being useful in practicing the processes of the invention are believed to be surface proteins present on cells, such as cancerous cells. Precisely how such cancer-linked proteins are used in the processes of the invention may thus differ depending on the therapeutic approach used. For example, cell-surface proteins, such as receptors, are desirable targets for cytotoxic antibodies that can be generated against the polypeptides disclosed herein.

The present invention relates to an isolated polypeptide, encoded by one of the polynucleotide transcripts disclosed herein, comprising an amino acid sequence homologous to the amino acid sequence of SEQ ID NO: 4, wherein any difference between amino acid sequence in the isolated polypeptide and the sequence of SEQ ID NO: 4 is due solely to conservative amino acid substitutions and wherein said isolated polypeptide comprises at least one immunogenic fragment. In a preferred embodiment, the present invention encompasses an isolated polypeptide comprising the amino acid sequence of SEQ ID NO: 4.

Methods of producing recombinant cells and vectors useful in preparing the polynucleotides and polypeptides disclosed herein are well known to those skilled in the molecular biology art. See, for example, Sambrook, et al., Molecular Cloning: A Laboratory Manual, Second Edition, Cold Spring Harbor, N.Y., (1989), Wu et al., Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference.

In one aspect, the present invention relates to a process for identifying an agent that modulates the activity of a cancer-related gene comprising:

(a) contacting a test compound with a cell that expresses a gene that corresponds to the polynucleotide having the sequence of SEQ ID NO: 3; and

(b) determining a difference in expression of said gene relative to when said contacting does not occur, wherein a said change in expression indicates modulation of said gene,

thereby identifying said test compound as an agent that modulates the activity of a cancer-related gene.

In specific embodiments of such process the cell is a cancer cell and the difference in expression is a decrease in expression. Such polynucleotides may also include those that have the sequence of SEQ ID NO: 3.

In another aspect, the present invention relates to a process for identifying an anti-neoplastic agent comprising contacting a cell exhibiting neoplastic activity with a compound first identified as a cancer related gene modulator using an assay process disclosed herein and detecting a decrease in said neoplastic activity after said contacting compared to when said contacting does not occur. Such neoplastic activity may include accelerated cellular replication and/or metastasis, and the decrease in neoplastic activity preferably results from the death of the cell.

The present invention also relates to a process for identifying an anti-neoplastic agent comprising administering to an animal exhibiting a cancer condition an effective amount of an agent first identified according to a process of one of one of the assays disclosed according to the invention and detecting a decrease in said cancerous condition.

In specific embodiments of the present invention, the genes useful for the invention comprise genes that correspond to the polynucleotide having the sequence of SEQ ID NO: 3, or may comprise the sequence of the cDNA (SEQ ID NO: 3) disclosed herein.

In accordance with the present invention, such assays rely on methods of determining the activity of the gene in question. Such assays are advantageously based on model cellular systems using cancer cell lines, primary cancer cells, or cancerous tissue samples that are maintained in growth medium and treated with compounds at a single concentration or at a range of concentrations. At specific times after treatment, cellular RNAs are conveniently isolated from the treated cells or tissues, which RNAs are indicative of expression of selected genes. The cellular RNA is then divided and subjected to differential analysis that detects the presence and/or quantity of specific RNA transcripts, which transcripts may then be amplified for detection purposes using standard methodologies, such as, for example, reverse transcriptase polymerase chain reaction (RT-PCR), etc. The presence or absence, or concentration levels, of specific RNA transcripts are determined from these measurements. The polynucleotide sequences disclosed herein are readily used as probes for the detection of such RNA transcripts and thus the measurement of gene activity and expression.

The polynucleotides of the invention can include fully operational genes with attendant control or regulatory sequences or merely a polynucleotide sequence encoding the corresponding polypeptide or an active fragment or analog thereof.

Because expression of the polynucleotide sequences disclosed herein are specific to the cancerous state, useful gene modulation is downward modulation, so that, as a result of exposure to an anti-neoplastic agent identified by the screening assays herein, the corresponding gene of the cancerous cell is expressed at a lower level (or not expressed at all) when exposed to the agent as compared to the expression when not exposed to the agent. For example, the gene sequence disclosed herein (SEQ ID NO: 3) correspond to a gene expressed at a higher level in cells of breast cancer than in normal breast cells. Thus, where said chemical agent causes this gene of the tested cell to be expressed at a lower level than the same genes of the reference, this is indicative of downward modulation and indicates that the chemical agent to be tested has anti-neoplastic activity.

In carrying out the assays disclosed herein, relative anti-neoplastic activity may be ascertained by the extent to which a given chemical agent modulates the expression of genes present in a cancerous cell. Thus, a first chemical agent that modulates the expression of a gene associated with the cancerous state (i.e., a gene corresponding to the polynucleotide transcript of SEQ ID NO: 3) to a larger degree than a second chemical agent tested by the assays of the invention is thereby deemed to have higher, or more desirable, or more advantageous, anti-neoplastic activity than said second chemical agent.

The gene expression to be measured is commonly assayed using RNA expression as an indicator. Thus, the greater the level of RNA (for example, messenger RNA or mRNA) detected the higher the level of expression of the corresponding gene. Thus, gene expression, either absolute or relative, is determined by the relative expression of the RNAs encoded by such genes.

RNA may be isolated from samples in a variety of ways, including lysis and denaturation with a phenolic solution containing a chaotropic agent (e.g., trizol) followed by isopropanol precipitation, ethanol wash, and resuspension in aqueous solution; or lysis and denaturation followed by isolation on solid support, such as a Qiagen resin and reconstitution in aqueous solution; or lysis and denaturation in non-phenolic, aqueous solutions followed by enzymatic conversion of RNA to DNA template copies.

Normally, prior to applying the processes of the invention, steady state RNA expression levels for the genes, and sets of genes, disclosed herein will have been obtained. It is the steady state level of such expression that is affected by potential anti-neoplastic agents as determined herein. Such steady state levels of expression are easily determined by any methods that are sensitive, specific and accurate. Such methods include, but are in no way limited to, real time quantitative polymerase chain reaction (PCR), for example, using a Perkin-Elmer 7700 sequence detection system with gene specific primer probe combinations as designed using any of several commercially available software packages, such as Primer Express software, solid support based hybridization array technology using appropriate internal controls for quantitation, including filter, bead, or microchip based arrays, solid support based hybridization arrays using, for example, chemiluminescent, fluorescent, or electrochemical reaction based detection systems.

The gene expression indicative of a cancerous state need not be characteristic of every cell of a given tissue. Thus, the methods disclosed herein are useful for detecting the presence of a cancerous condition within a tissue where less than all cells exhibit the complete pattern. Thus, for example, a selected gene corresponding to the sequence of SEQ ID NO: 3, may be found, using appropriate probes, either DNA or RNA, to be present in as little as 60% of cells derived from a sample of tumorous, or malignant, tissue. In a highly preferred embodiment, such gene pattern is found to be present in at least 100% of cells drawn from a cancerous tissue and absent from at least 100% of a corresponding normal, non-cancerous, tissue sample.

Expression of a gene may be related to copy number, and changes in expression may be measured by determining copy number. Such change in gene copy number may be determined by determining a change in expression of messenger RNA encoded by a particular gene sequence, especially that of SEQ ID NO: 3. Also in accordance with the present invention, said gene may be a cancer initiating or facilitating gene. In carrying out the methods of the present invention, a cancer facilitating gene is a gene that, while not directly initiating tumor formation or growth, acts, such as through the actions of its expression product, to direct, enhance, or otherwise facilitate the progress of the cancerous condition, including where such gene acts against genes, or gene expression products, that would otherwise have the effect of decreasing tumor formation and/or growth.

Although the expression of a gene corresponding to the sequence of SEQ ID NO: 3 may be indicative of a cancerous status for a given cell, the mere presence of such gene may not alone be sufficient to achieve a malignant condition and thus the level of expression of such gene may also be a significant factor in determining the attainment of a cancerous state. Thus, it becomes essential to also determine the level of expression of a gene as disclosed herein, including substantially similar sequences, as a separate means of diagnosing the presence of a cancerous status for a given cell, groups of cells, or tissues, either in culture or in situ.

The level of expression of the polypeptide disclosed herein is also a measure of gene expression, such as the polypeptide having sequence identical, or similar to, SEQ ID NO: 4.

In accordance with the foregoing, the present invention specifically contemplates a method for determining the cancerous status of a cell to be tested, comprising determining the level of expression in said cell of a gene that includes the nucleotide sequence of SEQ ID NO: 3, including sequences substantially identical to said sequence, or characteristic fragments thereof, or the complements of any of these and then comparing said expression to that of a cell known to be non-cancerous whereby the difference in said expression indicates that said cell to be tested is cancerous.

In accordance with the invention, although gene expression for a gene that includes as a portion thereof the sequence of SEQ ID NO: 3, is preferably determined by use of a probe that is a fragment of such nucleotide sequence, it is to be understood that the probe may be formed from a different portion of the gene. Expression of the gene may be determined by use of a nucleotide probe that hybridizes to messenger RNA (mRNA) transcribed from a portion of the gene other than the specific nucleotide sequence disclosed herein.

It should be noted that there are a variety of different contexts in which genes have been evaluated as being involved in the cancerous process. Thus, some genes may be oncogenes and encode proteins that are directly involved in the cancerous process and thereby promote the occurrence of cancer in an animal. In addition, other genes may serve to suppress the cancerous state in a given cell or cell type and thereby work against a cancerous condition forming in an animal. Other genes may simply be involved either directly or indirectly in the cancerous process or condition and may serve in an ancillary capacity with respect to the cancerous state. All such types of genes are deemed with those to be determined in accordance with the invention as disclosed herein. Thus, the gene determined by said process of the invention may be an oncogene, or the gene determined by said process may be a cancer facilitating gene, the latter including a gene that directly or indirectly affects the cancerous process, either in the promotion of a cancerous condition or in facilitating the progress of cancerous growth or otherwise modulating the growth of cancer cells, either in vivo or ex vivo. In addition, the gene determined by said process may be a cancer suppressor gene, which gene works either directly or indirectly to suppress the initiation or progress of a cancerous condition. Such genes may work indirectly where their expression alters the activity of some other gene or gene expression product that is itself directly involved in initiating or facilitating the progress of a cancerous condition. For example, a gene that encodes a polypeptide, either wild or mutant in type, which polypeptide acts to suppress of tumor suppressor gene, or its expression product, will thereby act indirectly to promote tumor growth.

As noted previously, polynucleotides encoding the same protein as SEQ ID NO: 3, regardless of the percent identity of such sequences, are also specifically contemplated by any of the methods of the present invention that rely on any or all of said sequences, regardless of how they are otherwise described or limited. Thus, any such sequences are available for use in carrying out any of the methods disclosed according to the invention. Such sequences also include any open reading frames, as defined herein, present within the sequence of SEQ ID NO: 3.

Because a gene disclosed according to the invention “corresponds to” a polynucleotide having the sequence of SEQ ID NO: 3, said gene encodes an RNA (processed or unprocessed, including naturally occurring splice variants and alleles) that is at least 90% identical, preferably at least 95% identical, most preferably at least 98% identical to, and especially identical to, an RNA that would be encoded by, or be complementary to, such as by hybridization with, a polynucleotide having the indicated sequence. In addition, genes including sequences at least 90% identical to the sequence of SEQ ID NO: 3, preferably at least about 95% identical to such a sequence, more preferably at least about 98% identical to such sequence and most preferably comprising such sequence are specifically contemplated by all of the processes of the present invention. Sequences encoding the same proteins as any of these sequences, regardless of the percent identity of such sequences, are also specifically contemplated by any of the methods of the present invention that rely on any or all of said sequences, regardless of how they are otherwise described or limited. The polynucleotide sequences of the invention also include any open reading frames, as defined herein, present within the sequence of SEQ ID NO: 3.

The sequences disclosed herein may be genomic in nature and thus represent the sequence of an actual gene, such as a human gene, or may be a cDNA sequence derived from a messenger RNA (mRNA) and thus represent contiguous exonic sequences derived from a corresponding genomic sequence, or they may be wholly synthetic in origin for purposes of practicing the processes of the invention. Because of the processing that may take place in transforming the initial RNA transcript into the final mRNA, the sequences disclosed herein may represent less than the full genomic sequence. They may also represent sequences derived from ribosomal and transfer RNAs. Consequently, the gene as present in the cell (and representing the genomic sequence) and the polynucleotide transcripts disclosed herein, including cDNA sequences, may be identical or may be such that the cDNAs contain less than the full genomic sequence. Such genes and cDNA sequences are still considered “corresponding sequences” (as defined elsewhere herein) because they both encode the same or related RNA sequences (i.e., related in the sense of being splice variants or RNAs at different stages of processing). Thus, by way of non-limiting example only, a gene that encodes an RNA transcript, which is then processed into a shorter mRNA, is deemed to encode both such RNAs and therefore encodes an RNA complementary to (using the usual Watson-Crick complementarity rules), or that would otherwise be encoded by, a cDNA (for example, a sequence as disclosed herein). Thus, the sequences disclosed herein correspond to genes contained in the cancerous cells (here, breast cancer) and are used to determine gene activity or expression because they represent the same sequence or are complementary to RNAs encoded by the gene. Such a gene also includes different alleles and splice variants that may occur in the cells used in the methods of the invention, such as where recombinant cells are used to assay for anti-neoplastic agents and such cells have been engineered to express a polynucleotide as disclosed herein, including cells that have been engineered to express such polynucleotides at a higher level than is found in non-engineered cancerous cells or where such recombinant cells express such polynucleotides only after having been engineered to do so. Such engineering includes genetic engineering, such as where one or more of the polynucleotides disclosed herein has been inserted into the genome of such cell or is present in a vector.

Such cells, especially mammalian cells, may also be engineered to express on their surfaces a polypeptide of the invention for testing with antibodies or other agents capable of masking such polypeptides and thereby removing the cancerous nature of the cell. Such engineering includes both genetic engineering, where the genetic complement of the cells is engineered to express the polypeptide, as well as non-genetic engineering, whereby the cell has been physically manipulated to incorporate a polypeptide of the invention in its plasma membrane, such as by direct insertion using chemical and/or other agents to achieve this result.

In accordance with the foregoing, the present invention includes anti-cancer agents that are themselves either polypeptides, or small chemical entities, that affect the cancerous process, including initiation, suppression or facilitation of tumor growth, either in vivo or ex vivo. Said cancer modulating agent will have the effect of decreasing gene expression.

The present invention thus also relates to a method for treating cancer comprising contacting a cancerous cell with an agent having activity against an expression product encoded by a gene or polynucleotide sequence as disclosed herein, such as one having, or corresponding to, the nucleotide sequence of SEQ ID NO: 3. The present invention also relates to a process for treating cancer comprising contacting a cancerous cell with an agent having activity against an expression product encoded by a gene or polynucleotide sequence corresponding to SEQ ID. NO: 3. In one such embodiment, the cancerous cell is contacted in vivo. In another such embodiment, said agent has affinity for said expression product. In a preferred embodiment, such agent is an antibody disclosed herein, such as an antibody that is specific or selective for, or otherwise reacts with, a polypeptide of the invention. In a preferred embodiment, the expression product is a polypeptide incorporating the amino acid sequence of SEQ ID NO: 4.

The present invention is also directed to such uses of the compositions of polypeptides and antibodies disclosed herein. Such uses include a process for treating cancer in an animal afflicted therewith comprising administering to said animal an amount of an immunogenic composition of one or more of the polypeptides disclosed herein where such amount if an amount sufficient to elicit the production of cytotoxic T lymphocytes specific for a polypeptide of the invention, preferably a polypeptide incorporating a sequence of SEQ ID NO: 4. In a preferred embodiment, the animal to be so treated is a human patient.

The proteins encoded by the genes disclosed herein due to their expression, or elevated expression, in cancer cells, represent highly useful therapeutic targets for “targeted therapies” utilizing such affinity structures as, for example, antibodies coupled to some cytotoxic agent. In such methodology, it is advantageous that nothing need be known about the endogenous ligands or binding partners for such cell surface molecules. Rather, an antibody or equivalent molecule that can specifically recognize the cell surface molecule (which could include an artificial peptide, a surrogate ligand, and the like) that is coupled to some agent that can induce cell death or a block in cell cycling offers therapeutic promise against these proteins. Thus, such approaches include the use of so-called suicide “bullets” against intracellular proteins. For example, monoclonal antibodies may readily by produced by methods well known in the art, for example, the method of Kohler and Milstein (see: Nature, 256:495 (1975).

With the advent of methods of molecular biology and recombinant technology, it is now possible to produce antibody molecules by recombinant means and thereby generate gene sequences that code for specific amino acid sequences found in the polypeptide structure of the antibodies. Such antibodies can be produced by either cloning the gene sequences encoding the polypeptide chains of said antibodies or by direct synthesis of said polypeptide chains, with in vitro assembly of the synthesized chains to form active tetrameric (H₂L₂) structures with affinity for specific epitopes and antigenic determinants. This has permitted the ready production of antibodies having sequences characteristic of neutralizing antibodies from different species and sources.

Regardless of the source of the antibodies, or how they are recombinantly constructed, or, how they are synthesized, in vitro or in vivo, using transgenic animals, such as cows, goats: and sheep, using large cell cultures of laboratory or commercial size, in bioreactors or by direct chemical synthesis employing no living organisms at any stage of the process, all antibodies have a similar overall 3 dimensional structure. This structure is often given as H₂L₂ and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V”0 regions and are characterized by differences in amino acid sequence from antibodies of different antigenic specificity.

The variable regions of either H or L chains contains the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure.

The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The accepted CDR regions have been described by Kabat et al., J. Biol. Chem. 252:6609-6616 (1977).

In all mammalian species, antibody polypeptides contain constant (i.e., highly conserved) and variable regions, and, within the latter, there are the CDRs and the so-called “framework regions” made up of amino acid sequences within the variable region of the heavy or light chain but outside the CDRs.

The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain. Such antibodies may also include fragments, such as Fab and F(ab₂)′ fragments, capable of reacting with and binding to any of the polypeptides disclosed herein as being receptors.

In one aspect, the present invention relates to immunoglobulins, or antibodies, as described herein, that react with, especially where they are specific for, the polypeptides having amino acid sequences as disclosed herein, preferably those having the amino acid sequence of SEQ ID NO: 4. Such antibodies may commonly be in the form of a composition, especially a pharmaceutical composition. Such antibodies, by themselves, may have therapeutic value in that they are able to bind to, and thereby tie up, surface sites on cancerous cells. Where such sites have some type of function to perform (i.e., where they are surface enzymes, or channel structures, or structures that otherwise facilitate, actively or passively, the transport of nutrients and other vital materials to the cell. Such nutrients serve to facilitate the growth and replication of the cell and molecules that bind to such sites and thereby interfere with such activities can prove to have a therapeutic effect in that the result of such binding is to remove sources of nutrients from such cells, thereby interfering with growth and replication. In like manner, such binding may serve to remove vital enzyme activities from the cell's functional repertoire, thereby also interfering with viability and/or the ability of the cell to multiply or metastasize. In addition, by binding to such surface sites, the antibodies may serve to prevent the cells from reacting to environmental agents, such as cytokines and the like, that may facilitate growth, replication and metastasis, thereby further reducing the cancerous status of such cell and ameliorating the cancerous condition in a patient, even without proving fatal to the cell or cells so affected.

The methods of the present invention also include processes wherein the cancer cell is contacted in vivo as well as ex vivo with an agent that comprises a portion, or is part of an overall molecular structure, having affinity for an expression product of a gene corresponding to a polynucleotide sequence as disclosed herein, preferably where the expression product is a cell surface structure, most preferably a polypeptide as disclosed herein, such as one that comprises an amino acid sequence homologous to, or including, SEQ ID NO: 4. In one such embodiment, said portion having affinity for said expression product is an antibody, especially where said expression product is a polypeptide or oligopeptide or comprises an oligopeptide portion, or comprises a polypeptide.

In another aspect, the present invention also relates to an antibody that reacts with a polypeptide as disclosed herein, preferably a polypeptide comprising the amino acid sequence of SEQ ID NO: 4. Such an antibody may be polyclonal, monoclonal, recombinant or synthetic in origin. In one such embodiment, said antibody is associated, either covalently or non-covalently, with a cytotoxic agent, for example, an apoptotic agent. It is thus contemplated that the antibody acts a targeted vector for guiding an associated therapeutic agent to a cancerous cell, such as a cell expressing a polypeptide homologous to, if not identical to, a polypeptide as disclosed herein.

Where the cytotoxic agent is itself a polypeptide, said may be linked directly to an antibody specific for a surface target on a cancer cell, such as where the polypeptide represents an extension of the amino acid chain of the antibody. In alternative embodiments, such molecules may be covalently linked through a linker sequence of long or short duration, such as an amino acid sequence of 5 to 10 residues in length. Where the cytotoxic agents is some small organic molecule, such as a small organic compound, or some type of apoptotic agent, this may be covalently bonded to the antibody molecule or may be attached by some other type of non-covalent linkage, including hydrophobic and electrostatic linkages. Methods for forming such linkages, especially covalent linkages, are well known to those skilled in the art.

The antibodies disclosed herein may also serve as targeting vectors for much larger structures, such as liposomes. In one such embodiment, an antibody is part of, or otherwise linked to, or associated with, a membranous structure, preferably a liposome or possibly some type of cellular organelle, which acts as a reservoir for a cytotoxic agent, such as ricin. The antibody then acts to target said liposome to a cancerous tissue in an animal, whereupon the liposome provides a source of cytotoxic agents for localized treatment of a solid tumor or other type of neoplasm.

The present invention further encompasses an immunogenic composition comprising a polypeptide disclosed herein, as well as compositions formed using antibodies specific for these polypeptides.

Methods well known in the art for making formulations are found in, for example, Remington: The Science and Practice of Pharmacy, (19th ed.) Ed. A. R. Gennaro, 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for agonists of the invention include ethylenevinyl acetate copolymer particles, osmotic pumps, implantable infusion systems, and liposomes. Formulations for inhalation may contain excipients, or example, lactose, or may be aqueous solutions containing, for example, polyoxyethylene-9-lauryl ether, glycocholate and deoxycholate, or may be oily solutions for administration in the form of nasal drops, or as a gel. It should be noted that, where the therapeutic agent to be administered is an immunoconjugate, these sometimes contain chemical linkages that are somewhat labile in aqueous media and therefor must be stored prior to administration is a more stable environment, such as in the form of a lyophilized powder.

Such an agent can be a single molecular structure, comprising both affinity portion and anti-cancer activity portions, wherein said portions are derived from separate molecules, or molecular structures, possessing such activity when separated and wherein such agent has been formed by combining said portions into one larger molecular structure, such as where said portions are combined into the form of an adduct. Said anti-cancer and affinity portions may be joined covalently, such as in the form of a single polypeptide, or polypeptide-like, structure or may be joined non-covalently, such as by hydrophobic or electrostatic interactions, such structures having been formed by means well known in the chemical arts. Alternatively, the anti-cancer and affinity portions may be formed from separate domains of a single molecule that exhibits, as part of the same chemical structure, more than one activity wherein one of the activities is against cancer cells, or tumor formation or growth, and the other activity is affinity for an expression product produced by expression of genes related to the cancerous process or condition.

In one embodiment of the present invention, a chemical agent, such as a protein or other polypeptide, is joined to an agent, such as an antibody, having affinity for an expression product of a cancerous cell, such as a polypeptide or protein encoded by a gene related to the cancerous process, preferably a gene as disclosed herein according to the present invention, most preferably a polypeptide sequence disclosed herein. Thus, where the presence of said expression product is essential to tumor initiation and/or growth, binding of said agent to said expression product will have the effect of negating said tumor promoting activity. In one such embodiment, said agent is an apoptosis-inducing agent that induces cell suicide, thereby killing the cancer cell and halting tumor growth.

Other genes within the cancer cell that are regulated in a manner similar to that of the genes disclosed herein and thus change their expression in a coordinated way in response to chemical compounds represent genes that are located within a common metabolic, signaling, physiological, or functional pathway so that by analyzing and identifying such commonly regulated groups of genes (groups that include the gene, or similar sequences, disclosed according to the invention, one can (a) assign known genes and novel genes to specific pathways and (b) identify specific functions and functional roles for novel genes that are grouped into pathways with genes for which their functions are already characterized or described. For example, one might identify a group of 10 genes, at least one of which is the gene as disclosed herein, that change expression in a coordinated fashion and for which the function of one, such as the polypeptide encoded by the sequence disclosed herein, is known then the other genes are thereby implicated in a similar function or pathway and may thus play a role in the cancer-initiating or cancer-facilitating process. In the same way, if a gene were found in normal cells but not in cancer cells, or happens to be expressed at a higher level in normal as opposed to cancer cells, then a similar conclusion may be drawn as to its involvement in cancer, or other diseases. Therefore, the processes disclosed according to the present invention at once provide a novel means of assigning function to genes, i.e. a novel method of functional genomics, and a means for identifying chemical compounds that have potential therapeutic effects on specific cellular pathways. Such chemical compounds may have therapeutic relevance to a variety of diseases outside of cancer as well, in cases where such diseases are known or are demonstrated to involve the specific cellular pathway that is affected.

The polypeptides disclosed herein, preferably that of SEQ ID NO: 4, also find use as vaccines in that, where the polypeptide represents a surface protein present on a cancer cell, such polypeptide may be administered to an animal, especially a human being, for purposes of activating cytotoxic T lymphocytes (CTLs) that will be specific for, and act to lyze, cancer cells in said animal. Where used as vaccines, such polypeptides are present in the form of a pharmaceutical composition. The present invention may also employ polypeptides that have the same, or similar, immunogenic character as the polypeptide of SEQ ID NO: 4 and thereby elicit the same, or similar, immunogenic response after administration to an animal, such as an animal at risk of developing cancer, or afflicted therewith. Thus, the polypeptides disclosed according to the invention will commonly find use as immunogenic compositions.

Expression of a gene corresponding to a polynucleotide disclosed herein, when in normal tissues, may indicate a predisposition towards development of breast cancer. The encoded polypeptide might then present a potentially useful cell surface target for therapeutic molecules such as cytolytic antibodies, or antibodies attached to cytotoxic, or cytolytic, agents.

The present invention specifically contemplates use of antibodies against the polypeptides encoded by the polynucleotides corresponding to the genes disclosed herein, whereby said antibodies are conjugates to one or more cytotoxic agents so that the antibodies serve to target the conjugated immunotoxins to a region of cancerous activity, such as a solid tumor. For many known cytotoxic agents, lack of selectivity has presented a drawback to their use as therapeutic agents in the treatment of malignancies. For example, the class of two-chain toxins, consisting of a binding subunit (or B-chain) linked to a toxic subunit (A-chain) are extremely cytotoxic. Thus, such agents as ricin, a protein isolated from castor beans, kills cells at very low concentrations (even less than 10⁻¹¹ M) by inactivating ribosomes in said cells (see, for example, Lord et al., Ricin: structure, mode of action, and some current applications. Faseb J, 8: 201-208 (1994), and Blättler et al., Realizing the full potential of immunotoxins. Cancer Cells, 1: 50-55 (1989)). While isolated A-chains of protein toxins that functionally resemble ricin A-chain are only weakly cytotoxic for intact cells (in the concentration range of 10⁻⁷ to 10⁻⁶ M), they are very potent cytotoxic agents inside the cells. Thus, a single molecule of the A-subunit of diphtheria toxin can kill a cell once inside (see: Yamaizumi et al., One molecule of diphtheria toxin fragment A introduced into a cell can kill the cell. Cell, 15: 245-250, 1978).

The present invention solves this selectivity problem by using antibodies specific for antigens present on cancer cells to target the cytotoxins to said cells. In addition, use of antibodies decreases toxicity because the antibodies are non-toxic until they reach the tumor and, because the cytotoxin is bound to the antibody, it is presented with less opportunity to cause damage to non-targeted tissues.

In addition, use of such antibodies alone can provide therapeutic effects on the tumor through the antibody-dependent cellular cytotoxic response (ADCC) and complement-mediated cell lysis mechanisms.

A number of recombinant immunotoxins (for example, consisting of Fv regions of cancer specific antibodies fused to truncated bacterial toxins) are well known (see, for example, Smyth et al., Specific targeting of chlorambucil to tumors with the use of monoclonal antibodies, J. Natl. Cancer Inst., 76(3):503-510 (1986); Cho et al., Single-chain Fv/folate conjugates mediate efficient lysis of folate-receptor-positive tumor cells, Bioconjug. Chem., 8(3):338-346 (1997)). As noted in the literature, these may contain, for example, a truncated version of Pseudomonas exotoxin as a toxic moiety but the toxin is modified in such a manner that by itself it does not bind to normal human cells, but it retains all other functions of cytotoxicity. Here, recombinant antibody fragments target the modified toxin to cancer cells which are killed, such as by direct inhibition of protein synthesis, or by concomitant induction of apoptosis. Cells that are not recognized by the antibody fragment, because they do not carry the cancer antigen, are not affected. Good activity and specificity has been observed for many recombinant immunotoxins in in vitro assays using cultured cancer cells as well as in animal tumor models. Ongoing clinical trials provide examples where the promising pre-clinical data correlate with successful results in experimental cancer therapy. (see, for example, Brinkmann U., Recombinant antibody fragments and immunotoxin fusions for cancer therapy, In Vivo (2000) 14:21-27).

While the safety of employing immunoconjugates in humans has been established, in vivo therapeutic results have been less impressive. Because clinical use of mouse MAbs in humans is limited by the development of a foreign anti-globulin immune response by the human host, genetically engineered chimeric human-mouse MAbs have been developed by replacing the mouse Fc region with the human constant region. In other cases, the mouse antibodies have been “humanized” by replacing the framework regions of variable domains of rodent antibodies by their human equivalents. Such humanized and engineered antibodies can even be structurally arranged to have specificities and effector functions determined by design and which characteristics do not appear in nature. The development of bispecific antibodies, having different binding ends so that more than one antigenic site can be bound, have proven useful in targeting cancer cells. Thus, such antibody specificity has been improved by chemical coupling to various agents such as bacterial or plant toxins, radionuclides or cytotoxic drugs and other agents. (see, for example, Bodey, B. et al). Genetically engineered monoclonal antibodies for direct anti-neoplastic treatment and cancer cell specific delivery of chemotherapeutic agents. Curr Pharm Des (2000) February; 6(3):261-76). See also, Garnett, M. C., Targeted drug conjugates: principles and progress. Adv. Drug Deliv. Rev. (2001 Dec. 17) 53(2):171-216; Brinkmann et al., Recombinant immunotoxins for cancer therapy. Expert Opin Biol Ther. (2001) 1(4):693-702.

Among the cytotoxic agents specifically contemplated for use as immunoconjugates according to the present invention are Calicheamicin, a highly toxic enediyne antibiotic isolated from Micromonospora echinospora ssp. Calichensis, and which binds to the minor groove of DNA to induce double strand breaks and cell death (see: Lee et al., Calicheamicins, a novel family of antitumor antibiotics. 1. Chemistry and partial structure of calichemicin g₁. J Am Chem Soc, 09: 3464-3466 (1987); Zein et al., Calicheamicin gamma 1I: an antitumor antibiotic that cleaves double-stranded DNA site specifically, Science, 240: 1198-1201 (1988)). Useful derivatives of the calicheamicins include mylotarg and 138H11-Camθ. Mylotarg is an immunoconjugate of a humanized anti-CD33 antibody (CD33 being found in leukemic cells of most patients with acute myeloid leukemia) and N-acetyl gamma colicheamicin dimethyl hydrazide, the latter of which is readily coupled to an antibody of the present invention (in place of the anti-CD33 but which can also be humanized by substitution of human framework regions into the antibody during production as described elsewhere herein) to form an immunoconjugate of the invention. (see: Hamann et al. Gemtuzumab Ozogamicin, A Potent and Selective Anti-CD33 Antibody-Calicheamicin Conjugate for Treatment of Acute Myeloid Leukemia, Bioconjug. Chem. 13, 47-58 (2002)) For use with 138H11-Camθ, 138H11 is an anti-γ-glutamyl transferase antibody coupled to theta calicheamicin through a disulfide linkage and found useful in vitro against cultured renal cell carcinoma cells. (see: Knoll et al., Targeted therapy of experimental renal cell carcinoma with a novel conjugate of monoclonal antibody 138H11 and calicheamicin θ₁^{I}, Cancer Res, 60: 6089-6094 (2000) The same linkage may be utilized to link this cytotoxic agent to an antibody of the present invention, thereby forming a targeting structure for breast cancer cells.

Also useful in forming the immunoconjugates of the invention is DC1, a disulfide-containing analog of adozelesin, that kills cells by binding to the minor groove of DNA, followed by alkylation of adenine bases. Adozelesin is a structural analog of CC-1065, an anti-tumor antibiotic isolated from microbial fermentation of Streptomyces zelensis, and is about 1,000 fold more toxic to cultured cell lines that other DNA interacting agents, such as cis-platin and doxorubicin. This agent is readily linked to antibodies through the disulfide bond of adozelesin. (see: Chari et al., Enhancement of the selectivity and antitumor efficacy of a CC-1065 analogue through immunoconjugate formation, Cancer Res, 55: 4079-4084 (1995)).

Maytansine, a highly cytotoxic microtubular inhibitor isolated from the shrub Maytenus serrata found to have little value in human clinical trials, is much more effective in its derivatized form, denoted DM1, containing a disulfide bond to facilitate linkage to antibodies, is up to 10-fold more cytotoxic (see: Chari et al., Immunoconjugates containing novel maytansinoids: promising anticancer drugs, Cancer Res, 52: 127-131 (1992)). These same in vitro studies showed that up to four DM1 molecules could be linked to a single immunoglobulin without destroying the binding affinity. Such conjugates have been used against breast cancer antigens, such as the neu/HER2/erbB-2 antigen. (see: Goldmacher et al., Immunogen, Inc., (2002) in press); also see Liu, C. et al., Eradication of large colon tumor xenografts by targeted delivery of maytansinoids, Proc. Natl. Acad. Sci. USA, 93, 8618-8623 (1996)). For example, Liu et al. (1996) describes formation of an immunoconjugate of the maytansinoid cytotoxin DM1 and C242 antibody, a murine IgG1 immunoglobulin, available from Pharmacia and which has affinity for a mucin-like glycoprotein variably expressed by human colorectal cancers. The latter immunoconjugate was prepared according to Chari et al., Cancer Res., 52:127-131 (1992) and was found to be highly cytotoxic against cultured colon cancer cells as well as showing anti-tumor effects in vivo in mice bearing subcutaneous COLO 205 human colon tumor xenografts using doses well below the maximum tolerated dose.

In addition, there are a variety of protein toxins (cytotoxic proteins), which include a number of different classes, such as those that inhibit protein synthesis: ribosome-inactivating proteins of plant origin, such as ricin, abrin, gelonin, and a number of others, and bacterial toxins such as pseudomonas exotoxin and diphtheria toxin.

Another useful class is the one including taxol, taxotere, and taxoids. Specific examples include paclitaxel (taxol), its analog docetaxel (taxotere), and derivatives thereof. The first two are clinical drugs used in treating a number of tumors while the taxoids act to induce cell death by inhibiting the de-polymerization of tubulin. Such agents are readily linked to antibodies through disulfide bonds without disadvantageous effects on binding specificity.

In one instance, a truncated Pseudomonas exotoxin was fused to an anti-CD22 variable fragment and used successfully to treat patients with chemotherapy-resistant hairy-cell leukemia. (see: Kreitman et al., Efficacy of the anti-CD22 recombinant immunotoxin BL22 in chemotherapy-resistant hairy-cell leukemia, N Engl J Med, 345: 241-247 (2001)) Conversely, the cancer-linked peptides of the present invention offer the opportunity to prepare antibodies, recombinant or otherwise, against the appropriate antigens to target solid tumors, preferably those of malignancies of breast tissue, using the same or similar cytotoxic conjugates. Thus, many of the previously used immunoconjugates have been formed using antibodies against general antigenic sites linked to cancers whereas the antibodies formed using the peptides disclosed herein are more specific and target the antibody-cytotoxic agent to a particular tissue or organ, thus further reducing toxicity and other undesirable side effects.

In addition, the immunoconjugates formed using the antibodies prepared against the cancer-linked antigens disclosed herein can be formed by any type of chemical coupling. Thus, the cytotoxic agent of choice, along with the immunoglobulin, can be coupled by any type of chemical linkage, covalent or non-covalent, including electrostatic linkage, to form the immunoconjugates of the present invention.

When used as immunoconjugates, the antitumor agents of the present invention represent a class of pro-drugs that are relatively non-toxic when first administered to an animal (due mostly to the stability of the immunoconjugate), such as a human patient, but which are targeted by the conjugated immunoglobulin to a cancer cell where they then exhibit good toxicity. The tumor-related, associated, or linked, antigens, preferably those presented herein, serve as targets for the antibodies (monoclonal, recombinant, and the like) specific for said antigens. The end result is the release of active cytotoxic agent inside the cell after binding of the immunoglobulin portion of the immunoconjugate.

The cited references describe a number of useful procedures for the chemical linkage of cytotoxic agents to immunoglobulins and the disclosures of all such references cited herein are hereby incorporated by reference in their entirety. For other reviews see Ghetie et al., Immunotoxins in the therapy of cancer: from bench to clinic, Pharmacol Ther, 63: 209-234 (1994), Pietersz et al. The use of monoclonal antibody immunoconjugates in cancer therapy, Adv Exp Med Biol, 353:169-179 (1994), and Pietersz, G. A. The linkage of cytotoxic drugs to monoclonal antibodies for the treatment of cancer, Bioconjug Chem, 1:89-95 (1990).

Thus, the present invention provides highly useful cancer-associated antigens for generation of antibodies for linkage to a number of different cytotoxic agents which are already known to have some in vitro toxicity and possess chemical groups available for linkage to antibodies.

The present invention also relates to a process that comprises a method for producing a product, such as by generating test data to facilitate identification of such product, comprising identifying an agent according to one of the disclosed processes for identifying such an agent (i.e., the therapeutic agents identified according to the assay procedures disclosed herein) wherein said product is the data collected with respect to said agent as a result of said identification process, or assay, and wherein said data is sufficient to convey the chemical character and/or structure and/or properties of said agent. For example, the present invention specifically contemplates a situation whereby a user of an assay of the invention may use the assay to screen for compounds having the desired enzyme modulating activity and, having identified the compound, then conveys that information (i.e., information as to structure, dosage, etc) to another user who then utilizes the information to reproduce the agent and administer it for therapeutic or research purposes according to the invention. For example, the user of the assay (user 1) may screen a number of test compounds without knowing the structure or identity of the compounds (such as where a number of code numbers are used the first user is simply given samples labeled with said code numbers) and, after performing the screening process, using one or more assay processes of the present invention, then imparts to a second user (user 2), verbally or in writing or some equivalent fashion, sufficient information to identify the compounds having a particular modulating activity (for example, the code number with the corresponding results). This transmission of information from user 1 to user 2 is specifically contemplated by the present invention.

In accorance with the foregoing, the present invention relates to a method for producing test data with respect to the gene modulating activity of a compound comprising:

(a) contacting a compound with a cell containing a polynucleotide comprising a nucleotide sequence corresponding to a gene whose expression is increased in a cancerous cell over that in a non-cancerous cell and under conditions wherein said polynucleotide is being expressed,

(b) determining a change in expression of polynucleotides as a result of said contacting, and

(c) producing test data with respect to the gene modulating activity of said compound based on a decrease in the expression of the determined gene whose expression is otherwise increased in a cancerous cell over that in a non-cancerous cell indicating gene modulating activity.

It should be cautioned that, in carrying out the procedures of the present invention as disclosed herein, whether to form immunoconjugates or screen for other antitumor agents using the genes and polypeptides disclosed herein, any reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The present invention will now be further described by way of the following non-limiting example. In applying the disclosure of the example, it should be kept clearly in mind that other and different embodiments of the methods disclosed according to the present invention will no doubt suggest themselves to those of skill in the relevant art. The following example shows how a potential anti-neoplastic agent may be identified using one or more of the genes disclosed herein.

### EXAMPLE

### Determination of Gene Inhibitory Activity of an Anti-Neoplastic Agent

SW480 cells are grown to a density of 10⁵ cells/cm² in Leibovitz's L-15 medium supplemented with 2 mM L-glutamine (90%) and 10% fetal bovine serum. The cells are collected after treatment with 0.25% trypsin, 0.02% EDTA at 37° C. for 2 to 5 minutes. The trypsinized cells are then diluted with 30 ml growth medium and plated at a density of 50,000 cells per well in a 96 well plate (100 μl/well). The following day, cells are treated with either compound buffer alone, or compound buffer containing a chemical agent to be tested, for 24 hours. The media is then removed, the cells lysed and the RNA recovered using the RNAeasy reagents and protocol obtained from Qiagen. RNA is quantitated and 10 ng of sample in 1 μl are added to 24 μl of Taqman reaction mix containing 1×PCR buffer, RNAsin, reverse transcriptase, nucleoside triphosphates, amplitaq gold, tween 20, glycerol, bovine serum albumin (BSA) and specific PCR primers and probes for a reference gene (18S RNA) and a test gene (Gene X). Reverse transcription is then carried out at 48° C. for 30 minutes. The sample is then applied to a Perlin Elmer 7700 sequence detector and heat denatured for 10 minutes at 95° C. Amplification is performed through 40 cycles using 15 seconds annealing at 60° C. followed by a 60 second extension at 72° C. and 30 second denaturation at 95° C. Data files are then captured and the data analyzed with the appropriate baseline windows and thresholds.

The quantitative difference between the target and reference gene is then calculated and a relative expression value determined for all of the samples used. In this way, the ability of a chemotherapeutic agent to effectively and selectively reduce the activity of a cancer-specific gene is readily ascertained. The overall expression of the cancer-specific gene, as modulated by one chemical agent relative to another, is also determined. Chemical agents having the most effect in reducing gene activity are thereby identified as the most anti-neoplastic.

### REFERENCES

Walter A. Blättler and Ravi Chari: Drugs to enhance the therapeutic potency of anti-cancer antibodies: antibody-drug conjugates as tumor-activated prodrugs. In AnticancerAgents—Frontiers in Cancer Chemotherapy (Iwao Ojima, Gregory D. Vite, Karl-Heinz Altmann, Eds.), American Chemical Society, pp. 317-338 (2001).
Dan L. Longo, Patricia L. Duffey, John G. Gribben, Elaine S. Jaffe, Brendan D. Curti, Barry L. Gause, John E. Janik, Virginia M. Braman, Dixie Esseltine, Wyndham H. Wilson, Dwight Kaufman, Robert E. Wiftes, Lee M. Nadler, and Walter J. Urba: Combination chemotherapy followed by an Immunotoxin (Anti-B4-blocked Ricin) in patients with indolent lymphoma: results of a Phase II study. Cancer J. 6, 146-150 (2000).
Walter A. Blättler and John M. Lambert: Preclinical immunotoxin development. In Monoclonal Antibody-Based Therapy of Cancer (M. Grossbard, Ed.), Marcel Dekker, Inc. NY, N.Y., pp. 1-22 (1998).
Ravi V. J. Chari: Targeted delivery of chemotherapeutics: tumor-activated prodrug therapy. In Advanced Drug Delivery Reviews, Elsevier Science B.V., pp. 89-104 (1998).
David T. Scadden, David P. Schenkein, Zale Bemstein, Barry Luskey, John Doweiko, Anil Tulpule, and Alexandra M. Levine: Immunotoxin combined with chemotherapy for patients with AIDS-related Non-Hodgkin's Lymphoma. Cancer 83, 2580-2587 (1998).
Changnian Liu and Ravi V J Chari: The development of antibody delivery systems to target cancer with highly potent maytansinoids. Exp. Opi. Invest Drugs 6,169-172 (1997).
A. C. Goulet, Viktor S. Goldmacher, John M. Lambert, C. Baron, Dennis C. Roy and E. Kouassi: Conjugation of blocked ricin to an anti-CD19 monoclonal antibody increases antibody-induced cell calcium mobilization and CD19 internalization. Blood 90, 2364-2375 (1997).
Changnian Liu, John M. Lambert, Beverly A. Teicher, Walter A. Blättler, and Rosemary O'Connor: Cure of multidrug-resistant human B-cell lymphoma xenografts by combinations of anti-B4-blocked ricin and chemotherapeutic drugs. Blood 87, 3892-3898 (1996).
Rajeeva Singh, Lana Kats, Walter A. Blättler, and John M. Lambert: Formation of N-Substituted 2-Iminothiolanes when amino groups in proteins and peptides are modified by 2-Iminothiolane. Anal. Biochem. 236, 114-125 (1996).
Changnian Liu, B. Mitra Tadayoni, Lizabeth A. Bourret, Kristin M. Mattocks, Susan M. Derr, Wayne C. Widdison, Nancy L. Kedersha, Pamela D. Ariniello,
Victor S. Goldmacher, John M. Lambert, Walter A. Blättler, and Ravi V. J. Chari: Eradication of large colon tumor xenografts by targeted delivery of maytansinoids. Proc. Natl. Acad. Sci. USA 93, 8618-8623 (1996).
Denis C. Roy, Sophie Ouellet, Christiane Le Houiller, Pamela D. Ariniello, Claude Perreault and John M. Lambert: Elimination of neuroblastoma and small-cell lung cancer cells with an anti-neural cell adhesion molecule immunotoxin. J. Natl. Cancer Inst. 88, 1136-1145 (1996).
Walter A. Blättler, Ravi V. J. Chari and John M. Lambert: Immunoconjugates. In Cancer Therapeutics: Experimental and Clinical Agents. (B. Teicher, Ed.), Humana Press, Totowa, N.J., pp. 371-394 (1996).
Michael L Grossbard, John M. Lambert, Victor S. Goldmacher, Arnold S. Freedman, Jeanne Kinsella, Danny P. Ducello, Susan N. Rabinowe, Laura Elisea, Felice Carol, James A. Taylor, Walter A. Blättler, Carol L. Epstein, and Lee M. Nadler: Anti-B4-blocked Ricin: A phase I trial of 7 day continuous infusion in patients with B-cell neoplasms. J. Clin. Oncol. 11, 726-737 (1993).
Michael L. Grossbard, John G. Gribben, Arnold S. Freedman, John M. Lambert, Jeanne Kinsella, Susan N. Rabinowe, Laura Eliseo, James A. Taylor, Walter A. Blättler, Carol L. Epstein, and Lee M. Nadler: Adjuvant immunotoxin therapy with anti-B4-blocked ricin following autologous bone marrow transplantation for patients with B-cell Non-Hodgkin's lymphoma. Blood 81, 2263-2271 (1993).
Sudhir A. Shah, Patricia M. Halloran, Cynthia A. Ferris, Beth A. Levine, Lizabeth A. Bourret, Victor S. Goldmacher, and Walter A. Blättler: Anti-B4-blocked Ricin immunotoxin shows therapeutic efficacy in four different SCID mouse tumor models. Cancer Res. 53, 1360-1367 (1993).
Ravi V. J. Chari, Bridget A. Martell, Jonathan L. Gross, Sherilyn B. Cook, Sudhir A. Shah, Walter A. Blättler, Sara J. McKenzie, and Victor S. Goldmacher: Immunoconjugates containing hovel maytansinoids: promising anti-cancer drugs. Cancer Res. 52, 127-131 (1992).
John M. Lambert, Peter D. Senter, Annie Yau-Young, Walter A. Blättler, and Victor S. Goldmacher: Purified immunotoxins that are reactive with human lymphoid cells. J. Biol. Chem. 250, 12035-12041 (1985).

What is claimed is:

1. A process for identifying an agent that modulates the activity of a cancer-related gene comprising:
(a) contacting a test compound with a cell that expresses a gene that corresponds to the polynucleotide having the sequence of SEQ ID NO: 3; and
(b) determining a difference in expression of said gene relative to when said contacting does not occur, wherein a said change in expression indicates modulation of said gene,
thereby identifying said test compound as an agent that modulates the activity of a cancer-related gene.

2. The process of claim 1 wherein said gene has the sequence of SEQ ID NO: 3.

3. The process of claim 1 or 2 wherein the cell is a cancer cell and the difference in expression is a decrease in expression.

4. The process of claim 3 wherein said cancer cell is a breast cancer cell.

5. A process for identifying an anti-neoplastic agent comprising contacting a cell exhibiting neoplastic activity with a compound first identified as a cancer related gene modulator using a process of claim 1 and detecting a decrease in said neoplastic activity after said contacting compared to when said contacting does not occur.

6. The process of claim 5 wherein said neoplastic activity is accelerated cellular replication.

7. The process of claim 5 wherein said decrease in neoplastic activity results from the death of the cell.

8. A process for identifying an anti-neoplastic agent comprising administering to an animal exhibiting a cancer condition an effective amount of an agent first identified according to a process of claim 1 and detecting a decrease in said cancerous condition.

9. A process for determining the cancerous status of a cell, comprising determining an increase in the level of expression in said cell of a gene that corresponds to a polynucleotide having the sequence of SEQ ID NO: 3 wherein an elevated expression relative to a known non-cancerous cell indicates a cancerous state or potentially cancerous state.

10. The process of claim 9 wherein said elevated expression is due to an increased copy number.

11. An isolated polypeptide comprising an amino acid sequence homologous to the amino acid sequence of SEQ ID NO: 4 wherein any difference between said amino acid sequence and the sequence of SEQ ID NO: 4 is due solely to conservative amino acid substitutions and wherein said isolated polypeptide comprises at least one immunogenic fragment.

12. An isolated polypeptide comprising the amino acid sequence of SEQ ID NO: 4.

13. An antibody that reacts with a polypeptide comprising the amino acid sequence of SEQ ID NO: 4.

14. The antibody of claim 13 wherein said antibody is a recombinant antibody.

15. The antibody of claim 13 wherein said antibody is a synthetic antibody.

16. The antibody of claim 13 wherein said antibody is a humanized antibody.

17. An immunoconjugate comprising the antibody of claim 13 and a cytotoxic agent.

18. The antibody of claim 17 wherein said cytotoxic agent is a member selected from the group consisting of a calicheamicin, a maytansinoid, an adozelesin, a cytotoxic protein, a taxol, a taxotere, a taxoid and DC1.

19. The immunoconjugate of claim 18 wherein said calicheamicin is calicheamicin γ₁^{I}, N-acetyl gamma calicheamicin dimethyl hydrazide or calicheamicin θ₁^{I}.

20. The immunoconjugate of claim 18 wherein said maytansinoid is DM1.

21. The immunoconjugate of claim 18 wherein said cytotoxic protein is ricin, abrin, gelonin, pseudomonas exotoxin or diphtheria toxin.

22. The immunoconjugate of claim 18 wherein said taxol is paclitaxel.

23. The immunoconjugate of claim 18 wherein said taxotere is docetaxel.

24. A process for treating cancer comprising contacting a cancerous cell in vivo with an agent having activity against an expression product encoded by the gene sequence of SEQ ID NO: 3.

25. The process of claim 24 wherein said agent is an antibody of claim 13-16.

26. The process of claim 24 wherein said agent is an immunoconjugate of claim 17.

27. An immunogenic composition comprising a polypeptide of claim 11.

28. An immunogenic composition comprising a polypeptide of claim 12.

29. The process of claim 24 wherein said cancer is breast cancer.

30. A process for treating cancer in an animal afflicted therewith comprising administering to said animal an amount of an immunogenic composition of claim 27 sufficient to elicit the production of cytotoxic T lymphocytes specific for the polypeptide of claim 11.

31. A process for treating cancer in an animal afflicted therewith comprising administering to said animal an amount of an immunogenic composition of claim 28 sufficient to elicit the production of cytotoxic T lymphocytes specific for the polypeptide of claim 12.

32. A process for treating a cancerous condition in an animal afflicted therewith comprising administering to said animal a therapeutically effective amount of an agent first identified as having anti-neoplastic activity using the process of claim 8.

33. A process for protecting an animal against cancer comprising administering to an animal at risk of developing cancer a therapeutically effective amount of an agent first identified as having anti-neoplastic activity using the process of claim 8.

34. The process of claim 30, 31, 32 or 33 wherein said animal is a human being.

35. The process of claim 30, 31, 32 or 33 wherein said cancer is breast cancer.

36. A method for producing test data with respect to the gene modulating activity of a compound comprising:
(a) contacting a compound with a cell containing a polynucleotide comprising a nucleotide sequence corresponding to a gene whose expression is increased in a cancerous cell over that in a non-cancerous cell and under conditions wherein said polynucleotide is being expressed,
(b) determining a change in expression of polynucleotides as a result of said contacting, and
(c) producing test data with respect to the gene modulating activity of said compound based on a decrease in the expression of the determined gene whose expression is otherwise increased in a cancerous cell over that in a non-cancerous cell indicating gene modulating activity.


SEQUENCE LISTING (4 sequences)

SEQ ID NO: 1 - DNA, 629 bp, Homo sapiens
  misc_feature (1)..(629): n=a, t, g, or c
tgtggcatattatttttattatggggaaagataactgagaataaagctatcatgcagatatttgcagagataaaagtaatgcagatactgagtggagttttgatcaaactatgcttgaaagccactctaccactagttacacaaaccaataatttcccttcgcagtggaagtcagcttgagttttttcaggtgtttttgtgggtttcaccagatacagcaaagaaattaaaattactgttaatggatgtcaaaaccagtcagaagtatcctaagttatataatttgtcaaacaaccatatacatatattttgtattatatttatccttttgttcttcctttggtaggaaaattgtctcattaattcttatacgaaaggacttaaaattagcaaactttttttgcaaacacatggattccattcttggacttgaggacaacttgacgaacaggctggggaggccttgagtggtctggagccagcttgaagcggagcagagttaatgccactgccactntacactcaattatggcaaaatgctgcccaatgcagttccttaatccagctgagaatggtatgaaggcatanggatgtatnntttcagaaatttccctggagaatctcagggg

SEQ ID NO: 2 - protein, 760 aa, Homo sapiens
GGAAAGATAACTGAGAATAAAGCTATCATGCAGATATTTGCAGAGATAAAAGTAATGCAGATACTGAGTGGAGTTTTGATCAAACTATGCTTGAAAGCCACTCTACCACTAGTTACACAAACCAATAATTTCCCTTCGCAGTGGAAGTCAGCTTGAGTTTTTTCAGGTGTTTTTGTGGGTTTCACCAGATACAGCAAAGAAATTAAAATTACTGTTAATGGATGTCAAAACCAGTCAGAAGTATCCTAAGTTATATAATTTGTCAAACAACCATATACATATATTTTGTATTATATTTATCCTTTTGTTCTTCCTTTGGTAGGAAAATTGTCTCATTAATTCTTATACGAAAGGACTTAAAATTAGCAAACTTTTTTTGCAAACACATGGATTCCATTCTTGGACTTGAGGACAACTTGACGAACAGGCTGGGGAGGCCTTGAGTGGTCTGGAGCCAGCTTGAAGCGGAGCAGAGTTAATGCCACTGCCACTTTACACTCAATTATGGCAAAATGCTGCCCAATGCAGTTCCTTAATCCAGCTGAGAATGGTATGAAGGCATAAGGATGTATTTTTCAGATTTTCCCTTGAGAATCTCAAGCGTTAAAGACCTGAGGGGTCTCCCAGAAATAGGGGTTTGGGTGAAGAGCCCACATATTGATAACACAGTATTCCTGCCCGTACGAGCGTCCATCTGGAAAAGGAATGGGTTTGCGAGTACACGGATATTGTTACTTACCGTGCGTANAAGCGGAGGATA

SEQ ID NO: 3 - DNA, 1906 bp, Artificial
  cDNA
agaatggagccctcctggcttcaggaactcatggctcaccccttcttgctgctgatcctcctctgcatgtctctgctgctgtttcaggtaatcaggttgtaccagaggaggagatggatgatcagagccctgcacctgtttcctgcaccccctgcccactggttctatggccacaaggagttttacccagtaaaggagtttgaggtgtatcataagctgatggaaaaatacccatgtgctgttcccttgtgggttggaccctttacgatgttcttcagtgtccatgacccagactatgccaagattctcctgaaaagacaagatcccaaaagtgctgttagccacaaaatccttgaatcctgggttggtcgaggacttgtgaccctggatggttctaaatggaaaaagcaccgccagattgtgaaacctggcttcaacatcagcattctgaaaatattcatcaccatgatgtctgagagtgttcggatgatgctgaacaaatgggaggaacacattgcccaaaactcacgtctggagctctttcaacatgtctccctgatgaccctggacagcatcatgaagtgtgccttcagccaccagggcagcatccagttggacagtaccctggactcatacctgaaagcagtgttcaaccttagcaaaatctccaaccagcgcatgaacaattttctacatcacaacgacctggttttcaaattcagctctcaaggccaaatcttttctaaatttaaccaagaacttcatcagttcacagagaaagtaatccaggaccggaaggagtctcttaaggataagctaaaacaagatactactcagaaaaggcgctgggattttctggacatacttttgagtgccaaaagcgaaaacaccaaagatttctctgaagcagatctccaggctgaagtgaaaacgttcatgtttgcaggacatgacaccacatccagtgctatctcctggatcctttactgcttggcaaagtaccctgagcatcagcagagatgccgagatgaaatcagggaactcctaggggatgggtcttctattacctgggaacacctgagccagatgccttacaccacgatgtgcatcaaggaatgcctccgcctctacgcaccggtagtaaacatatcccggttactcgacaaacccatcacctttccagatggacgctccttacctgcaggaataactgtgtttatcaatatttgggctcttcaccacaacccctatttctgggaagaccctcaggtctttaaccccttgagattctccagggaaaattctgaaaaaatacatccctatgccttcataccattctcagctggattaaggaactgcattgggcagcattttgccataattgagtgtaaagtggcagtggcattaactctgctccgcttcaagctggctccagaccactcaaggcctccccagcctgttcgtcaagttgtcctcaagtccaagaatggaatccacgtgtttgcaaaaaaagtttgctaattttaagtcctttcgtataagaattaatgagacaattttcctaccaaaggaagaacaaaaggataaatataatacaaaatatatgtatatggttgtttgacaaattatataacttaggatacttctgactggttttgacatccattaacagtaattttaatttctttgctgtatctggtgaaacccacaaaaacacctgaaaaaactcaagctgacttccactgcgaagggaaatattggtttgtgtaactagtggtagagtggctttcaagcatagtttgatcaaaactccactcagtatctgcattacttttatctctgcaaatatctgcatgatagctttattctcagttatctttccccataataaaaataatatgccaca

SEQ ID NO: 4 - protein, 505 aa, Artificial
  Putative protein derived from cDNA.
MEPSWLQELMAHPFLLLILLCMSLLLFQVIRLYQRRRWMIRALHLFPAPPAHWFYGHKEFYPVKEFEVYHKLMEKYPCAVPLWVGPFTMFFSVHDPDYAKILLKRQDPKSAVSHKILESWVGRGLVTLDGSKWKKHRQIVKPGFNISILKIFITMMSESVRMMLNKWEEHIAQNSRLELFQHVSLMTLDSIMKCAFSHQGSIQLDSTLDSYLKAVFNLSKISNQRMNNFLHHNDLVFKFSSQGQIFSKFNQELHQFTEKVIQDRKESLKDKLKQDTTQKRRWDFLDILLSAKSENTKDFSEADLQAEVKTFMFAGHDTTSSAISWILYCLAKYPEHQQRCRDEIRELLGDGSSITWEHLSQMPYTTMCIKECLRLYAPVVNISRLLDKPITFPDGRSLPAGITVFINIWALHHNPYFWEDPQVFNPLRFSRENSEKIHPYAFIPFSAGLRNCIGQHFAIIECKVAVALTLLRFKLAPDHSRPPQPVRQVVLKSKNGIHVFAKKVC